(19) United States
(12) Patent Application Publication
(10) Pub. No.: US 2008/0071336 A1
(43) Pub. Date: Mar. 20, 2008

(54) THERAPEUTIC HYGIENE ARTICLE

(76) Inventor: Janelle Merriman, Redondo Beach, CA (US)

Correspondence Address: IPLA P.A., 3580 WILSHIRE BLVD., 17TH FLOOR, LOS ANGELES, CA, 90010, US

(21) Appl. No.: 11/533,617

(22) Filed: Sep. 20, 2006

(51) Int. Cl. A61F 7/12; A61F 7/00

(52) U.S. Cl. 607/113; 607/114

(57) ABSTRACT

A therapeutic hygiene article for postpartum vaginal relief comprises a pouch for insertion in a user's vaginal orifice and a cooling pack formed within the pouch.

### BACKGROUND OF THE INVENTION

The present invention relates to a therapeutic nursing article and, more particularly, to an innovatively improved therapeutic hygiene article incorporated in a pad with a cooling mechanism to thereby provide to its wearer an instant relief of pain and discomfort resulting from postpartum vaginal irritations.

An estimated one-third of U.S. adults or some 80 million people suffer hemorrhoids, pain and bleeding related thereto. Some 200 million dollars a year is spent on products to treat them. Chronic hemorrhoids affect some 9 million Americans, and can turn sitting, walking, sneezing, laughing, and defecating into extremely painful propositions. For swollen, inflamed hemorrhoids, medical doctors usually recommend an ice pack, saying “The cooling is soothing, and helps shrink swollen tissue”. To relieve pain, you can apply an ice pack (with a soft covering) to the affected area several times a day. Ice may help decrease swelling and discomfort. Some women find cold compresses saturated with witch hazel to be soothing.

Applicant believes no ready made ice cooling products for postpartum vaginal relief are available on the market, that serve a multitude of purposes in bodily cavity irritation relief. Therefore, a need on the market is an article that provides instant pain relief and alleviates discomfort by shrinking swollen tissue. Another need is ice cooling products that can be worn like a sanitary napkin that serves as an irritation relief pad with therapeutic effects.

### SUMMARY OF THE INVENTION

The present invention is contrived to overcome the conventional disadvantages. Accordingly, an objective of the present invention is to provide a therapeutic hygiene article incorporated in a pad with a cooling mechanism to thereby provide to its wearer an instant relief of pain and discomfort resulting from postpartum vaginal irritations.

Another objective is to improve therapeutic effects by adopting witch hazel smeared in a pouch covering a cooling pack.

A further objective is to employ a refrigeration-free gel sheet that stays more than four hours cooled without refrigeration, thereby reserving usability and marketability.

To achieve these and other objectives, a therapeutic hygiene article for postpartum vaginal relief comprises a pouch for insertion in a user's vaginal orifice, and a cooling pack formed within the pouch. In an embodiment, the hygiene article further comprises an adhesive fabric with an inner surface thereof attached to the pouch and an outer surface thereof detachably attached to a user's underwear.

The pouch of the hygiene article may be provided with lanolin for moisturizing effect and soaked with an aloe solution for soothing effect. Selectively, the pouch may be further soaked with an anti-inflammatory agent. The cooling pack is preferably a refrigeration-free gel sheet. Further, the pouch functions to absorb bodily wastes. The inner surface of the adhesive fabric is incorporated to and unitary with the pouch. The cooling pack is reusable so the cooling pack can be freezer cooled.

In a preferred embodiment, the therapeutic hygiene article for postpartum vaginal relief comprises a pouch for insertion in a user's vaginal orifice where the pouch is soaked with witch hazel, a cooling pack formed within the pouch, and an adhesive fabric with an inner surface thereof adhered to the pouch and an outer surface thereof detachably attached to a user's underwear. Menthol may be selectively soaked to the pouch. The hygiene article may extend to cover the user's anal orifice.

Although the present invention is briefly summarized, the full understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a plan view of a therapeutic hygiene article according to a preferred embodiment of the present invention;

FIG. 2 is a cross sectional view taken along II-II in FIG. 1;

FIG. 3 is an application view showing a user's application of the therapeutic hygiene article for hemorrhoid pain relief;

FIG. 4 is a view showing another embodiment of the present invention; and

FIGS. 5 and 6 are schematic plan views each showing variations of the therapeutic hygiene article according to the present invention.

### DETAILED DESCRIPTION OF THE PREFERRED EMBODIMENTS

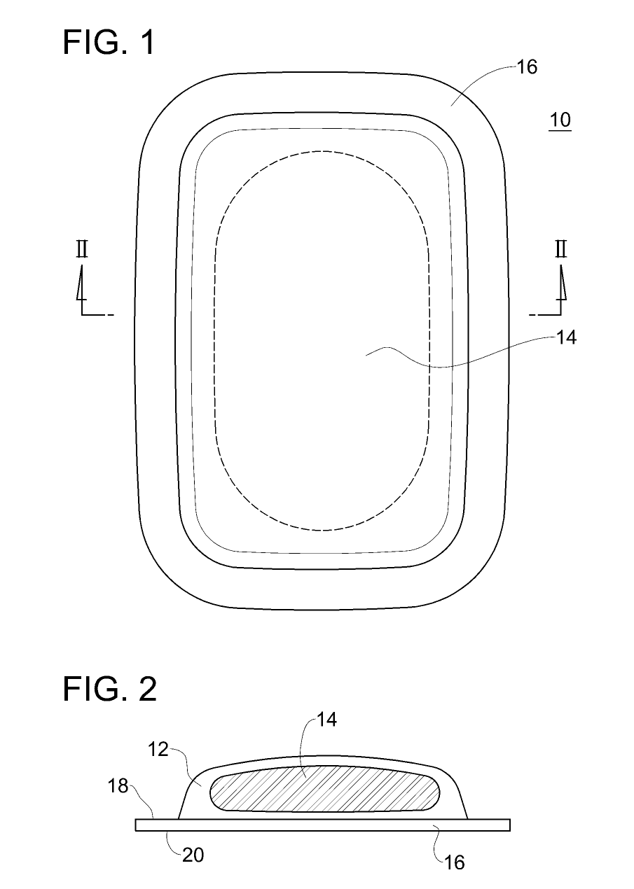

As shown in FIGS. 1 and 2, a therapeutic hygiene article 10 for postpartum vaginal relief comprises a pouch 12 for insertion in a user's vagina, and a cooling pack 14. The cooling pack 14 is formed within the pouch 12. To improve a user's satisfaction, lanolin may be added to the pouch 12 for moisturizing and emulsifying effects. Lanolin serves as an emollient with moisturizing properties and an emulsifier with high water absorbing capabilities.

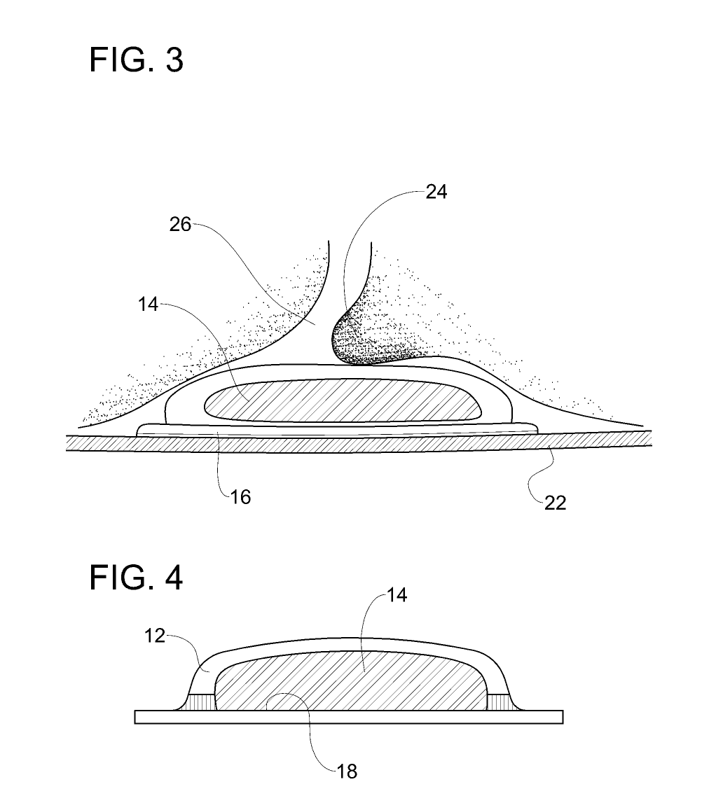

In an embodiment, the hygiene article 10 further comprises an adhesive fabric 16 where an inner surface 18 of the adhesive fabric 16 is selectively attached to the pouch 12 and an outer surface 20 of the adhesive fabric 16 is detachably attached to a user's underwear 22 shown in FIG. 3. The hygiene article 10 is preferably shaped in pad like a women's sanitary napkin. The pouch 12 may has an opening to facilitate insertion of the cooling pack 14. In a preferred version, the hygiene article 10 may extend to cover the user's anal cavity.

The pouch 12 is formed of fabric material to absorb liquids. The pouch 12 of the hygiene article 10 may be soaked with an aloe solution. Aloe is known to detoxify and eliminate toxins in the body. The pouch 12 soaked in the aloe solution serves to eliminate toxins when wrapped over to an irritation 24 as further shown in FIG. 3.

In the hygiene article 10, the pouch 12 may be selectively soaked with an anti-inflammatory agent. In an embodiment, the pouch 12 may be soaked with both an aloe solution and an anti-inflammatory agent. The anti-inflammatory agent soaked in the pouch 12 may be non-steroidal. Here, ‘soaked’ includes smearing, spraying and other ways of applying the ingredients for soothing of discomfort and healing.

The cooling pack 14 of the hygiene article 10 can be a gel type. Specifically, it is recommended that the cooling pack 14 is replaced with a refrigeration-free gel sheet that stays cool for at least four hours without refrigeration. This type of cooling gel sheet helps maintain the hygiene article 10 substantially as thin as women's sanitary napkin on the market. Depending upon requirements, the user may refrigerate the gel sheet type cooling pack 14 for further cooling. The gel type cooling sheet without requiring refrigeration has been introduced by Kobayashi Pharmaceutical in Japan.

The pouch 12 introduced in this hygiene article 10 functions to effectively absorb bodily wastes that may come out from around the user's bodily irritation 24 in a bodily cavity 26 as illustrated in FIG. 3. The bodily cavity 26 may refer to either a vaginal or an anal orifice. The pouch 12 is provided with sufficient cotton warps to maintain a required absorbent property in its use.

Selectively, the inner surface 18 of the adhesive fabric 16 is incorporated to and unitary with the pouch 12. In this construction, as shown in FIG. 4, the inner surface 18 of the adhesive fabric 16 serves as a side surface forming the pouch 12 so as to also enable forming of the pouch 12 in a detachable attachment system where the adhesive fabric 16 may be detached from the pouch 12 to insert the cooling pack 14 that can be reusable. When the cooling pack 14 is provided in a reusable format, it can be easily cooled in a freezer to an extent where the user requires.

In a preferred version, the cooling pack 14 may become cooled at break or bending. For example, a room temperature cooling pack on a table or in a package turns to an instantly cooled pack at the user's reshaping or breaking of the flat pack to an application stage.

For a better performance, the pouch 12 may be soaked with witch hazel which is an extract of the leaves and bark of the Hamamelis virginnia plant and known as an effective astringent. The astringent effect of witch hazel may serve to shrink hemorrhoids 24. Witch hazel is also known to dry out spots, reduce oil on the skin, and soothe bruises and sprains. Consequently, the soaking application of witch hazel in the pouch 12 substantially relieves and soothes postpartum vaginal irritations 24 and discomfort thereby.

In a preferred mode, the pouch 12 soaked with witch hazel may be further soaked with menthol which is known as the main element in essential oil of peppermint. Menthol is used in body care products for its soothing properties. The application of menthol to the pouch 12 serves to further relieve pain and discomfort related to postpartum irritations after vaginal birth.

In an embodiment, the hygiene article 10 can be positioned to cover simultaneously both the vagina and anal cavity. The adhesive fabric may be used to hold the article 10 in position. Alternately, the therapeutic hygiene article may be folded and inserted directly into the vagina, where pressure from the sides of the vagina will keep the article 10 in place. Discomfort from the stretching and irritation of the skin due to child birth is effectively relieved by insertion of the hygiene device 10 into the vagina, such that the article 10 is in optimal direct contact with the area of discomfort. The article 10 may be reusable or disposable depending on market needs.

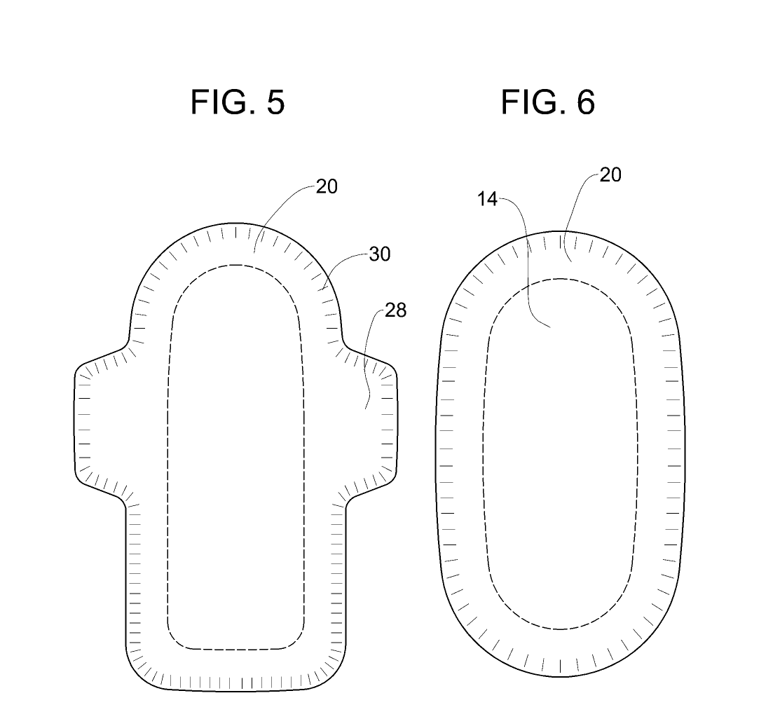

FIGS. 5 and 6 shows variations of the hygiene article 10. The article 10 may be provided with wings 28 to improve adhesivity to the user's underwear 22. The hygiene article 10 may be primarily formed in a substantially rectangular shape as shown back in FIG. 1 with a still preferred oval shape shown in FIG. 6. It is recommended that adhesives 30 can be evenly distributed over the outer surface 20 of the fabric 16. Selectively, the adhesives 30 may be formed along periphery of the outer surface 20 of the fabric 16.

Medicated ingredients in the article 10 such as witch hazel and menthol will soothe, cool and relieve suffering by bodily orifice irritations. Both products 10 either reusable or disposable have different shapes for a gentle contour of ones' buttocks and extra comfort to suit all needs. These shapes include oval, square and round as further shown in FIGS. 5 and 6. Further, the soft thinly padded-medical absorbable and non-stick material forming the pouch 12 will maximize comfort. When folded, these packs 10 fit securely into the contour of ones' buttocks.

The article 10 with a refrigeration-required gel type cooling pack 14 is easily cooled in the freezer, its non-toxic thermal reusable gel pack slides into a soft medicated absorbable and non-stick material pouch for maximum comfort. It is preferred that this medicated absorbable material for the pouch 12 is made of polymer with a soft stay dry lining to assure the consumer that it will absorb any light liquid from condensation, or blood from the problem area.

The medicated solutions on the pouches 12 will come two different ways. One way, the pouch will be soaked with three different medicated ingredients. In a preferred mode, the user can choose to have witch hazel, aloe and an anti inflammatory/anti-itch liquid. Selectively, the consumer may also buy the pouch un-medicated and simply spray on the preferred solution as needed. After each use, discard the pouch and use a new pouch when needed. A disposable hygiene article 10 is easily cooled in the freezer and contains a medicated liquid in both of the above forms, for its icing purpose. The article 10 may be designed for use at room temperature. In this application, once the pack is bent, a pop will occur chemically causing instant cooling to relieve postpartum vaginal suffering.

For both disposable packs, the consumer can choose to buy the pre-soaked medicated solution pouches, or spray on the three ingredients stated above, per individual needs. They soothe, cool and provide immediate relief that millions of Americans need when suffering from postpartum discomfort. Absorbable soft material for the pouch 12 will serve as a cool satisfaction to many other situations as well. An example of an important need will be a postpartum aide in vaginal births. The soft absorbable material will collect blood as the cooling ingredient soothes the vaginal area.

As discussed above, the therapeutic hygiene article 10 is provided with a therapeutic effect combined with a cooling mechanism to thereby provide to its wearer an instant relief of pain and discomfort resulting from postpartum vaginal irritations. The therapeutic effects are realized by adopting witch hazel smeared in the pouch 12 covering the cooling pack 14. Further, the application of the refrigeration-free gel sheet that stays more than four hours cooled without refrigeration serves to maximize usability and marketability of the hygiene article 10.

Although the invention has been described in considerable detail with reference to certain preferred versions thereof, other versions are possible by converting the aforementioned construction. For example, the hygiene article 10 can be used for hemorrhoid pain relief for those free from postpartum vaginal irritation. Therefore, the scope of the invention shall not be limited by the specification specified above and the appended claims.

1. A therapeutic hygiene article for anal relief, comprising:
a pouch for placement upon a user's anal orifice; and
a cooling pack formed within the pouch.

2. The hygiene article of claim 1, wherein the pouch has lanolin.

3. The hygiene article of claim 1, wherein the pouch is sprayed with an aloe solution.

4. The hygiene article of claim 1, wherein the pouch is smeared with an anti-inflammatory agent.

5. The hygiene article of claim 1, wherein the cooling pack is a refrigeration-free gel sheet.

6. The hygiene article of claim 1, wherein the pouch is disposable and absorbs bodily wastes.

7. The hygiene article of claim 1, wherein the cooling pack is freezer cooled.

8. The hygiene article of claim 1, wherein the cooling pack is form from an absorbent material that accepts and retains water.

9. A therapeutic hygiene article for anal hemorrhoid relief, comprising:
a pouch for placement upon a anal user's orifice, the pouch being soaked with witch hazel; and
a cooling pack formed within the pouch.

10. The hygiene article of claim 9, wherein the pouch is provided with lanolin and further soaked with an aloe solution.

11. The hygiene article of claim 9, wherein the pouch is further soaked with an anti-inflammatory agent.

12. The hygiene article of claim 9, wherein the cooling pack is a refrigeration-free gel sheet.

13. The hygiene article of claim 9, wherein the pouch absorbs bodily wastes.

14. The hygiene article of claim 9, wherein the cooling pack is reusable.

15. The hygiene article of claim 9, wherein the cooling pack is formed from an absorbent material.

16. A therapeutic hygiene article for anal hemorrhoid postpartum vaginal relief, comprising:
a pouch for placement upon the anal and vaginal region, the pouch being sprayed with witch hazel and menthol;
a cooling pack formed within the pouch; and
an adhesive fabric with an inner surface thereof adhered to the pouch and an outer surface thereof detachably attached to the user's underwear.

17. The hygiene article of claim 16, wherein the pouch is further soaked with an aloe solution and an anti-inflammatory agent.

18. The hygiene article of claim 16, wherein the cooling pack is a refrigeration-free gel sheet.

19. The hygiene article of claim 16, wherein the inner surface of the adhesive fabric is incorporated to and unitary with the pouch.

20. The hygiene article of claim 16, wherein the cooling article pack is formed from an absorbent material and unitary with the pouch.
